(19) United States
(12) Patent Application Publication
(10) Pub. No.: US 2002/0157118 A1
(43) Pub. Date: Oct. 24, 2002

(54) GENETICALLY ENGINEERED ANIMALS CONTAINING NULL MUTATIONS IN THE NEUROFILAMENT GENES

(76) Inventors: ROBERT A. LAZZARINI, NEW YORK, NY (US); GREGORY ELDER, NEW YORK, NY (US)

Correspondence Address: BAKER & BOTTS, 30 ROCKEFELLER PLAZA, NEW YORK, NY, 10112

(21) Appl. No.: 09/338,033

(22) Filed: Jun. 22, 1999

Related U.S. Application Data
(63) Continuation of application No. PCT/US98/08877, filed on May 1, 1998.
(60) Provisional application No. 60/045,264, filed on May 1, 1997.

(51) Int. Cl. A01K 67/027; A01K 67/033

(52) U.S. Cl. 800/3; 800/12; 800/13; 800/14; 800/18; 800/21; 800/24; 800/25

(57) ABSTRACT

The present invention provides for genetically engineered non-human animals, including but not limited to mice, which lack one or more endogenous neurofilament gene. Human neurofilament genes may be introduced into such genetically non-human animals (referred to hereafter as “knockout animals”) to produce improved models of the physiology of human neurofilament proteins which may be used to study human neurofilament-associated neurodegenerative conditions.

This invention was made with government support under NIA grant #P50 AGO 5138-11 awarded by the National Institutes of Health. The government has certain rights in this invention.

### 1. INTRODUCTION

The present invention relates to the development of genetically engineered non-human animals which lack one or more endogenous neurofilament genes, for use as models for human neurodegenerative diseases. The invention further relates to the introduction of human neurofilament genes into such genetically engineered animals to produce improved model systems for the study of human neurodegenerative diseases. Such genetically engineered animals can be used to test new therapies, including pharmacological based therapies, aimed at preventing or treating human neurodegenerative diseases.

### 2. BACKGROUND OF THE INVENTION

Alzheimer's disease (AD) is a degenerative disorder of the central nervous system characterized clinically by progressive loss of memory along with other cognitive skills and associated with neuropathologic features including extracellular amyloid deposits and intraneuronal neurofibrillary tangles. Although the etiology of AD is likely multifactorial including both genetic and environmental factors some cases show a clear pattern of autosomal dominant transmission and are referred to as familial AD (FAD).

To date three genes involved in FAD have been identified. The first mutations were discovered in the amyloid protein precursor (APP) gene. Six different missense mutations in exons 16 and 17 of APP can cause either FAD or an inherited form of recurrent intracerebral hemorrhage (Chartier-Harlin et al., Nature, 1991, 353:844-846; Goate, A. et al., Nature, 1991, 349:704-706; Hendriks, L. et al., Nature Genetics, 1992, 1:218-221; Levy, E. et al., Science, 1990, 248:1124-1126; Mullan, M. et al., Nature Genetics, 1991, 1:345-347; Murrell, J. et al., Science, 1991, 254:97-99). A second FAD locus (AD3) was mapped by several groups to the region around chromosome 14_{q}24.3 (Schellenberg, G. D. et al., Science, 1992, 258:668-671; St. George-Hyslop, P. et al., Nature Genetics, 1992, 2:330-334; Van Broeckhoven, C. et al., Nature Genetics, 1992, 2: p. 335-339). Recently Sherrington et al. (Sherrington, R. et al., Nature, 1995, 375: 754-760) identified a minimal cosegregating unit containing the AD3 gene and using a direct cDNA selection approach isolated multiple transcripts encoded within this region. Five distinct missense mutations in one transcript (S182) cosegregated with early-onset FAD. Additional novel mutations were soon identified by other groups (Alzheimer's Disease Research Group, Nature Genetics, 1995, 11:219-222; Campion, D. et al., Human Molecular Genetics, 1995, 4:2373-2377; Cruts, M. et al., Human Molecular Genetics, 1995, 4:2363-2371; Sorbi, S. et al., Lancet, 1995, 346: 439-440; Tanahashi, H. et al., Lancet, 1995, 346:440) and to date more than 20 different missense mutations linked to FAD have been identified in a gene, now termed presenillin-I (PS-I) (Van Broeckhoven, C. Nature Genetics, 1994, 11: 230-232). Most recently a third gene responsible for FAD in certain families of German dissent (the Volga German kindreds) was identified on chromosome 1 (Rogaev, E. I. et al., Nature, 1995, 376:775-778; Levy-Lahad, E. et al., Science, 1995, 269: 970; Levy-Lahad, E. et al., Science, 1995. 269:973-977). Interestingly this gene, termed STM2, E5-1, or presenilin II (PS-II), shows substantial homology to the PS-I gene. Two missense mutations appeared responsible for the disease in these families Rogaev, E. I. et al., Nature, 1995, 376:775-778; Levy-Lahad, E. et al., Science, 1995, 269:973-977).

Three proteins termed the neurofilament (NF) triplet are the most prominent cytoskeletal components in large myelinated axons and are probably the most abundant and widely expressed of neuronal intermediate filament proteins. Neurofilaments are incorporated into a range of lesions found in neurodegenerative diseases including the neurofibrillary tangles occurring in AD and the intracellular inclusions found in amyotrophic lateral sclerosis (ALS). Despite their suspected importance in processes such as establishing axonal diameter and their potential importance in human neurodegenerative diseases, little is known about the function(s) of the NFs in general or the roles of several specialized domains found only in NFs. It is also not known to what extent the structural differences between rodent and human NFs translates into functional differences between the proteins.

Because there are substantial differences between human and mouse neurofilament proteins, wild type mice are not suitable for the study of the role of neurofilaments in human neurodegenerative conditions, such as AD and ALS.

### 3. SUMMARY OF THE INVENTION

The present invention provides for genetically engineered non-human animals, including but not limited to mice, which lack one or more endogenous neurofilament gene. Specifically, the engineered laboratory animals can be engineered to lack the light (NF-L), medium (NF-M) and/or heavy (NF-H) neurofilament genes (referred to hereafter as “knockout animals”). In addition, the invention further provides for the introduction of human neurofilament genes into such genetically non-human animals to produce improved animal models for studying the physiology of neurofilament proteins and neurofilament-associated neurodegenerative conditions.

The genetically engineered laboratory animals of the invention are expected to develop or be predisposed to developing neurodegenerative diseases or disorders based on the aberrant expression of neurofilament proteins within neurons. Thus, the present invention provides animal models of such diseases and disorders which can be used to screen for or test the effectiveness of therapeutic agents (e.g., potential therapeutic drugs) for their ability to prevent or treat neurodegenerative disorders.

### 4. DESCRIPTION OF THE FIGURES

FIG. 1. Map of murine NF(H) gene and targeting strategy for disruption of the mouse NF-H gene. The structure of endogenous mouse NF-H is shown in the top line. Exons are indicated by open boxes. A targeting vector designed to utilize positive and negative selection (Mansour, S. L. et al., Nature, 1988, 336:348-352) is shown in the center line. The PGK/Neo gene was inserted in a sense orientation between Not I and Xho I sites (nucleotides −15 and +207 in Shneidman et al. (Shneidman, P. S. et al., Mol. Brain Res., 1988, 4:217-231) in the first exon of mouse NF-H. The NF-H vector removes the initial ATG and the first 71 amino acids of the protein. It contains 2.1 kb of 5′ and 13.4 kb of 3′ homologous sequence and was linearized with Kpn I. The overall targeting frequency was approximately 1 in 200 clones.

FIG. 2A. Southern blot of DNA from ES cells with disrupted NF-H alleles. A Southern blot is shown of a targeted ES cell clone digested with Kpn I (K), Xba I (X), or Kpn I/Xbal double digest and probed with the Bam HI/Sac I fragment indicated in FIG. 1. Two clones show the expected wild type pattern (+/+) while the targeted clone (+/−) gives an additional Xba band and a 2.9 kb Kpn/Xba band (indicated by arrow) consistent with homologous recombination.

FIG. 2B. Southern blot of offspring from a heterozygous/heterozygous mating is shown. DNA was digested with Bam HI and probed with the Bam HI/Eco RI fragment shown above. Examples of wild type (+/−), heterozygous (+/−) and null mutant animals (−/−).

FIGS. 3A-3D. Cerebellum from a wild type (+/+) littermate (FIGS. 3A and 3B) or a homozygous (−/−) NF(H) knockout (FIGS. 3C and 3D) were double labeled by two color immunofluorescence (FITC and Texas red) with a rabbit anti-NF(L) antisera (FIGS. 3A and 3C) and a monoclonal rat-anti NF(H) antibody RMO24 (gift of Dr. Virginia Lee, FIGS. 3B and 3D). Neurofilament staining in the Purkinje cell layer is illustrated for both animals. As shown in the figure, the wild type (+/+) mouse exhibits strong immunostaining for both NF(L) and NF(H) while the homozygous mutant (−/−) exhibits no detectable NF(H) even though strong NF(L) staining is seen in the same section (C). The lack of NF(H) expression has also been documented by Western blotting.

FIG. 4A. Diameters of all myelinated axons were measured in L5 ventral roots of 4 month old animals (N=2 wild type, N=3 mutant). Data is presented on all axons greater than 2 μm in diameter. Note the marked reduction of axons greater than 6 μm in diameter in the mutant accompanied by an increase in smaller diameter fiber.

FIG. 4B. Axon diameters were measured in the sciatic nerve of a 2 month old wild type and mutant animal. Quantitation was performed by sampling every fifth myelinated axon in the largest trunk of a proximal portion of the nerve. Data is presented for all axons greater that 2 μm in diameter (N=374, wild type, 313 NF-M−/−). Note the reduced numbers of larger diameter axons in the null mutant accompanied by a shift to medium diameter fibers.

FIG. 4C. Axon sizes were measured in 1.9×10⁵ μm² area of the ventral medial portion of the third cervical segment. This region was chosen since comparable areas could be easily identified in different animals and because this region contains many large axons. Data is presented for all axons greater that 5 μm in diameter (N=311 for wild type and 620 for NF-H−/−) from a 2 month old wild type and mutant NF-H animal. Note the dramatic reduction in large diameter fibers accompanied by a shift to smaller diameter fibers in the null mutant.

FIG. 4D. Axon sizes were determined in the optic nerves of 2 month old wild type and NF-H null mutant animals. Quantitation was performed on electron micrographs of optic nerve by measuring every myelinated axon in four randomly selected fields (N=686, wild type, 741 NF-H−/−). Note the shift towards smaller diameter fibers in the mutant.

FIG. 5A. NFs were counted in myelinated axons of L5 ventral root axons of wild type and control animals. The number of NFs in each axon was plotted against axonal size (area in square microns). Note that in myelinated axons of similar size the wild type has slightly more NF's than the NF-H null mutant.

FIG. 5B. Microtubules were counted in the same axons as in FIG. 5A. No significant difference between mutant and control was found in the number of microtubules.

FIG. 5C. NF densities were determined using methods similar to those described by Price et al., (1988, J. Neurocytol. 17:55-62). A template of hexagons was applied over each electron micrograph and the number of NFs per hexagon counted in all hexagons which fell completely within axonal borders. Hexagons were excluded only if vesicular organelles filled more than approximately 10% of the hexagon. At least 300 hexagons (N=351 wild type, 357 NF-M mutant) each equivalent to an area of 0.10 sq. Microns were counted and a frequency distribution plot was generated showing the number of HFs per hexagon. Note the reduced density of NFs in the NF-H mutant.

FIGS. 6A-B. Interfilament spacing was analyzed in 10 mutant (range 1.88 to 11.08 square microns, average 5.29+/−3.64 S.D.) And 10 wild type (range 1.33 to 10.59, average 4.62+/−3.33) axons from the L5 ventral root. The position of all NFs is each axon cut in true cross section (N=4708 mutant and 4965 wild type) were determined and nearest neighbor distances computed. Note that although the decreased NF density in the mutant results in an increased average interfilament distance, the modal distance is similar in both mutant and control. FIG. 6B shows the values for the individual axons measured in FIG. 6A.

FIG. 7. Diagram of targeting strategy for disruption of the mouse NF-M gene. The structure of the endogenous mouse NF-M is shown in the top line. Exons are indicated by open boxes. A targeting vector designed to use positive and negative selection is shown in the center line. A PGK/Neo resistance gene was cloned in an anti-sense orientation and replaces an ˜800-bp SacI-EcoRI fragment that includes the last 93 bp of exon 1 and most of intron 1. It contains 2 kb of 5′ and 1.5 kb of 3′ homologous sequence and was linearized with HindIII. A map of the targeted recombinant gene is shown in the bottom line of the panel.

FIG. 8. Southern blot of DNA from mice with disrupted NF-M alleles. A Southern blot of tail DNA from offspring of a heterozyous/heterozyous mating is shown. Blotting was performed with the downstream HindIII probe indicated in FIG. 7. Successful targeting converts a wild-type (WT) 7.2-kb BamHI fragment to a 5.5-kb mutant (M) band. Examples of wild-type (+/+) heterozygous (+/−), and null mutant animals (−/−) are indicated.

FIG. 9. Rnase protection assay was performed with 25,000 CPM of an exon 3 murine NF-M probe (3′ to the neomycin resistance gene) and 5,000 CPM of a GAPDH probe. Protected fragments were separated as a double-stranded RNA on a 6% native polyacrylamide gel. Positions of the 240-bp GAPDH and 129-bp NF-M protected fragments are indicated. Lanes were hybridized with 10 μg of total brain RNA from an unrelated wild-type mouse (lane 2), a homozygous mutant (−/−, lane 3), and heterozygous (+/−,lane 4) or wild-type (+/+ lane 5) littermates.

FIG. 10. No detection of NF-M protein in NF-M null mice. Western blotting was performed with a polyclonal rabbit antiserum (NFM-N) raised against the head domain of NF-M. No full-length or truncated NF-M protein could be detected in the spinal cord of NF-M^{-/-}mice. Lower molecular weight bands in the wild-type (+/+) lane likely reflect degradation products.

FIG. 11. Quantitative Western blots of neocortex and spinal cord of NF-M heterozyous (+/−), NF-M null (−/−), and wild-type (+/+) mice. Each sample was loaded in triplicate. The NF-M immunoreactivities are decreased in both neocortex and spinal cord of the heterozyous mice and undetectable in the null mice. A concomitant decrease in NF-L is also observed in both neocortex and spinal cord of the heterozygous and null mice. NFHP- is measured with RmdO9, a mAb against poorly or nonphosphorylated NF-H epitopes; NFHP+++, with RM024 a mAb against the rod domain of NF-M; NF-L, with a polyclonal rabbit anti-NFL antiserum; TUB, with a mAb specific for β-tubulin. All animals were 3 mo old.

FIG. 12A. Quantitative RNase protection assays were performed on total brain RNA from two wild-type and two NF-M^{-/-} animals each 2 mo old. NF-L levels were normalized to the expression of β-actin. Data from three independent determinations for each sample is shown. Results are presented as arbitrary units with wild-type NF-L levels set as 100. NF-L levels in wild type were 100±6 (SEM) and in NF-M^{-/-} animals 111±11 (P=0.35, unpaired t test).

FIG. 12B. A sample RNase protection assay is shown, 5 μg of total brain RNA from a wild-type (lane 1) or NF-M null mutant (lane 2) were hybridized with 20,000 cpm of a mouse NF-L probe and 10,000 cpm of a murine β-actin probe. Protected fragments were separated as double stranded RNA on a 6% native polyacrylamide gel. Positions of the NF-L and actin bands are indicated.

FIG. 13A. Light microscopy of toluidine blue-stained L5 ventral roots from a 4-month old wild type and NF-M null mutant mouse. Note the reduced size of the NF-M mutant (−/−) root as well as the absence in the mutant of axons with calibers comparable to the largest present in the control.

FIG. 13B. Diameters of all mylinated axons were measured in L5 roots (n=4 wild type, n=3 mutant). Note the marked reduction of axons>8 μm in diameter in the mutant accompanied by an increase in smaller diameter fibers.

FIG. 13C. Axon diameters were measured in the sciatic nerve of a four month old wild type and mutant animal. Quantitation was performed by sampling every fifth myelinated axon in the largest trunk of a proximal portion of the nerve. Data is presented for all axons>2 μM in diameter (n=374, wild type, 313 NF-M−/−). Note the absence of any exons>9.0 μM in diameter in the null mutant accompanied by a shift towards a smaller diameter fibers.

FIG. 13D. Western Blot of neurofilament content in the ventral roots. Total protein recovered from one L5 ventral root was loaded per lane. The level of NF-L protein was decreased by about 50%, while an increase in the level of β-tubulin was observed.

FIG. 13E. Western Blot of neurofilament content in sciatic nerves. 10 μg of total protein was loaded per lane. The level of NF-L protein was decreased by about 50%, while an increase in the level of β-tubulin was observed.

FIG. 14A. Axonal calibers in the ventral spinal cord of wild type and NF-M mutant mice. Axon sizes were measured in a 1.9×10⁵ μm² area of the ventral medial portion of C3 (boxed area shown in FIG. 14B). Data is presented for all axons>5 μm in diameter (n=263 for wild type and 307 for NF-M−/−) from a 5-month old wild type and mutant NF-M animal. Note the dramatic reduction in large diameter fibers accompanied by a shift to smaller diameter fibers in the null mutant.

FIG. 14B. Light microscopy of a toluidine blue-stained section of ventral cervical cord (C3) from a wild type animal. Box indicates the region used to generate the frequency distributions shown in FIG. 14A. Bar, 50 μm.

FIG. 15A and 15B. Axon calibers in optic nerves. Electron micrographs from optic nerves of 5-mo-old NF-M null mutant (FIG. 15A) and wildtype (FIG. 15B) mice. Note the generally reduced size of myelinated axons in the NF-M animal. Bar, 3 μm.

FIG. 15C. Axon sizes were determined in the optic nerves of a 5-mold wild type and NF-M null mutant. Quantitation was performed by sampling every third myelinated axon in five randomly selected fields (n=193 wild type, 322 NF-M^{-/-}). Note the shift towards smaller diameter fibers in the mutant. Bar, 3 μm.

FIG. 16A and 16B. Appearance of neurofilaments in mice with an NF-M null mutation. NFs in axons of L5 ventral root are viewed in cross section and longitudinally (insets) from 4-mo-old NF-M null mutant (FIG. 16A) or wild-type mice (FIG. 16B). NFs (triangles) are reduced in the NF-M null mutant as compared with control, while microtubules (asterisks) are increased. Bar, 300 nm.

FIGS. 17A-17D. Neurofilament and microtubule content in NF-M-deficient animals. NFs were counted in the intemodal regions of L5 ventral root axons of 4-mo-old mutant and control animals. The number of NFs in each axon was plotted against axonal size (area in μm²). Note that in axons of similar size, the wild type has more NFs than the NF-M null mutant (FIG. 17A). NF densities were determined using methods similar to those described by Price et al. (35). A template of hexagons was applied over each electron micrograph and the number of NFs per hexagon counted. At least 300 hexagons (n=314 wild type, 322 NF-M mutant) each equivalent to an area of 0.10 μm² were counted and a frequency distribution plot was generated showing the number of NFs per hexagon. Note the dramatically reduced density of NFs in the NF-M mutant (FIG. 17B). Microtubules were counted in the same axons as in 17A. In contrast to NFs, axons in NF-M mutant animals have more microtubules than axons of comparable size in wild type (FIG. 17C). The ratio of microtubules (MT) to NFs is shown for the axons (FIG. 17D).

FIGS. 18A-18B. Interfilament spacing was analyzed in 10 mutant (range 0.78-8.72 μm², average 3.91±3.10 SD) and 10 wild-type (range 1.63-8.58, average 3.99±2.50) axons from the L5 ventral roots of 4-mo-old animals. The positions of all NFs in each axon (n=1,683 mutant and 4,709 wild type) were determined and nearest neighbor distances computed. Note that although the decreased NF density in the mutant results in an increased average interfilament distance, the modal distance is similar in both mutant and control. FIG. 18B. Values for the individual axons measured in A are shown.

FIG. 19. Diagram of Targeting Vector designed to create NF(M) and NF(L) double null mutants.

### 5. DETAILED DESCRIPTION OF THE INVENTION

The present invention relates to the development of genetically engineered non-human animals, including but not limited to mice, that lack one or more endogenous neurofilament genes. The invention further relates to the engineering of such animals to contain human neurofilament genes. In this regard, the animals of the invention may be genetically engineered to contain either wild type or mutant human neurofilament genes. Additionally, the present invention provides an improved animal model system for the study of human neurodegenerative and methods which utilize the animals of the present invention to screen for agents useful for treatment of neurodegenerative disorders.

### 5.1. Generation of Targeting Vectors

The targeting vectors of the present invention comprise nucleic acid molecules comprising the light (NF-L), medium (NF-M) and heavy (NF-H) neurofilament genes. The neurofilament genes may be derived from an animal of any species, which includes, but is not limited to mouse or human genes to generate model animals. The vectors of the present invention are useful for the production of genetically engineered laboratory animals whose germ cells and somatic cells contain mutations in the neurofilament genes. Nucleic acid molecules encoding the neurofilament genes are known in the art (see, for example, Myers et al., 1987, EMBO J., 6:1617-1626; Lees J. F., et al., 1988, EMBO J., 7:1947-1955; Lee, 1992, Mol. Brain Res. 15:76-84; Steinart, P. M. et al., Annu. Rev. Biochem. 57, 593-625; Julien et al., Mol. Brain Res. 1:243-250; Levy E. et al., 166:71-77; Carter, J.Biol.Chem., 1998, 273:5101-5108).

Those of ordinary skill in the art can obtain a nucleic acid molecule encoding the neurofilament gene of interest using standard procedures well known to those skilled in the art. Any DNA can be used as a source for the molecular cloning of the desired neurofilament genes. The DNA can be obtained by standard procedures known in the art such as cDNA cloning, genomic cloning, chemical synthesis, etc. (See, for example, Sambrook, 1989, Molecular Cloning, A Laboratory Manual, 2d Ed.); and Glover, D. M., 1985, DNA Cloning: A Practical Approach MRL Press, Ld, Oxford, U.K. Vol. I, II). Alternatively, polymerase chain reaction (PCR) can be used to amplify the desired neurofilament gene from a genomic or cDNA library using oligonucleotide primers representing known neurofilament sequences. Once the desired neurofilament gene has been isolated it can then be inserted into an appropriate targeting vector using any of the methods commonly employed and known in the art, such as those described in Ausubel et al., 1993, Current Protocols in Molecular Biology; and Kriegler, 1990, Gene Transfer and Expression).

To generate genetically engineered laboratory animals containing null mutations in the neurofilament genes, the neurofilament transgene contained in the targeting vector is an inactivated gene. Inactivation of the neurofilament gene may be accomplished using a variety of methods well known in the art for introducing mutations into a DNA molecule, including but not limited to, elimination of translation initiation site, creation of variations in the coding region of the gene including the insertion of missense or nonsense mutations, and insertions of heterologous DNA into the coding region of the neurofilament gene. In a preferred embodiment of the invention one or more selectable marker genes are inserted into the neurofilament gene to produce an inactive neurofilament transgene.

In a preferred embodiment of the invention, the targeting vectors are designed to utilize the double (positive/negative) selection procedure Mansour, S. L. et al., Nature, 1988, 336:348-352. In such vectors both the thymidine kinase (TK) gene and the neomycin (neo) resistance gene are included in the targeting vector. Using such targeting vectors, correctly targeted events will integrate only the neo gene, therefore, conferring G148 resistance to the cells (positive selection). Any random integration events will also have the TK gene integrated into the genome, allowing such integrates to convert the non-toxic analog FIAU into a lethal compound by the action of the TK gene product thus providing a negative selection for incorrect targeting events.

In addition, to ensure expression of the neurofilament transgene the neurofilament gene can be inserted into a targeting vector that contains the necessary regulatory elements for the transcription and translation of the inserted protein-coding sequence. As used herein, regulatory elements include but are not limited to inducible and non-inducible promoters, enhancers, operators, and other elements known to those of skill in the art that drive and regulate expression, and can be utilized without undue experimentation. The necessary transcriptional and translational signals can also be supplied by the native neurofilament genes and/or their flanking regions. For tissue specific expression of the neurofilament transgene, the coding portion of the gene can be ligated to a regulatory sequence which is capable of driving expression in a tissue specific manner. In order to overexpress the neurofilament gene sequence, the coding portion of the neurofilament gene sequence can be ligated to a regulatory sequence which is capable of driving high level gene expression.

Any of the methods previously described for the insertion of DNA fragments into a vector may be used to construct expression vectors containing a human neurofilament transgene consisting of appropriate transcriptional/translational control signals and the protein coding sequences. These methods may include in vitro recombinant DNA and synthetic techniques and in vivo recombinants (genetic recombination).

### 5.2. Generation of Genetically Engineered Animals

The present invention provides for genetically engineered non-human animals in which the neurofilament genes have been rendered biologically inactive. The invention further provides for genetically engineered non-human animals into which wild-type or mutant human neurofilament genes have been introduced. Methods for making such genetically engineered animals are known in the art and include, for example, pronuclear microinjection, retroviral mediated gene transfer into germ line cells, blastomere-embryo aggregation, gene targeting in embryonic stem cells, electroporation of embryos, nuclear transplantation, and spermatozoa-mediated transfer. Methods for generating genetically engineered animals are reviewed, for example by Pinkert et al.(1995, Transgenic Animal Modeling, in Molecular Biology and Biotechnology, Myers, ed., pp. 90-107), and numerous laboratory manuals including, for example, Hogan et al.(1994, Manipulating the Mouse Embryo: A Laboratory Manual, 2^{nd} edition), the disclosure of which is incorporated herein by reference.

Genetically engineered animals lacking neurofilament genes can be initially produced by promoting homologous recombination between the endogenous neurofilament gene in its chromosomal location and the neurofilament gene contained in the targeting vector. The neurofilament gene contained in the targeting vector has been rendered biologically inactive, preferably by insertion of a heterologous sequence, e.g., an antibiotic resistance gene, into the neurofilament gene. Alternatively, homologous recombination may be used to promote homologous recombination between an inactivated neurofilament gene in its chromosomal location and a wild type or mutant human neurofilament gene contained in a targeting vector.

In a preferred embodiment, homologous recombination is carried out by transforming embryo-derived stem cells (ES) with a vector containing an insertionally inactivated neurofilament gene, or a human neurofilament gene, such that homologous recombination occurs. The transformed ES cells are then injected into a blastocyst, and implanted into a foster mother, followed by the birth of the chimeric animal (“knockout animal”) in which a neurofilament gene has been inactivated (see Capecchi, 1989, Science 244:1288-1292).

DNA from the offspring can be isolated from tissue samples and analyzed to identify individual animals that carry the transgene. Detection of the transgene can be accomplished using for example Southern blot analysis, or alternatively, polymerase chain reaction utilizing primers specific for the specific neurofilament transgene. The absence of neurofilament RNA expression may be assayed using techniques, which include but are not limited to, Northern blot analysis of tissue samples obtained from animals, RNase protection studies or in situ hybridization. A variety of different methods may be used for detection of neurofilament protein including techniques known in the art for detection of proteins including, for example, immunoblotting, immunoprecipitation and immunohistochemical staining.

Once individual animals containing the transgene are identified, animals can be bred to other animals, containing different knockouts, for example, to generate genetically non-human animals with multiple inactivated genes. The genetically engineered laboratory animals may also be bred to animals with different inbred genetic backgrounds so as to examine effects of modifying alleles on the activity of the neurofilament transgene

In a specific embodiments of the invention, described in detail below, transgenic mice were produced by homologous recombination in embryonic stem cells to generate mice lacking the NF(H) and NF(M) neurofilament genes. Animals carrying null mutations in both genes could be produced by breeding an animal carrying a mutation in the NF(M) neurofilament gene with an animal carrying a mutation in the NF(H) neurofilament gene. Since the mouse NF(L) and NF(M) genes are located on the same chromosome a genetically engineered mouse can be generated with null mutations in both the NF(M) and NF(L) genes. Such a double knockout mouse can then be bred with a mouse containing a NF(H) knockout to produce an animal with null mutations in all three neurofilament genes. One or more human neurofilament genes, representing either the wild type or a mutant gene, may also be introduced as transgenes into the animals carrying null mutations in endogenous NF genes using the techniques described above.

### 5.3 Uses for the Genetically Engineered Animals

Neurofibrillary tangles and other types of neurofibrillary pathologies associated with neurodegenerative disorders are generally not seen in animals such as mice. Thus, a mouse containing human neurofilament genes provides a useful animal model system for determining if the structural differences between the human and mouse neurofilament proteins relates to an animal's propensity to develop pathologies associated with neurodegenerative disorders.

In addition, genetically engineered animals containing wild type, or mutant, human neurofilament genes may be bred to animals with different genetic backgrounds including, for example, animals containing mutations in genes known to play a role in neurodegenerative disorders, such as the APP gene or the presenilin genes. The effect of such mutant alleles on the activity of the human neurofilament genes may be examined in the bred animals.

The present invention further provides a method for identifying agents useful in the treatment or prevention of neurodegenerative disorders comprising administering the agent to the genetically engineered animal of the present invention and assessing the symptoms and progression of neurodegeneration in the animal, wherein an amelioration in symptoms or slowing of progression of neurodegeneration relative to untreated animals is indicative of an agent usefuil in the treatment or prevention of neurodegeneration. The agents to be tested may be administered to the animal by methods known to those of ordinary skill in the art and suitable for the selected agent. Possible administration routes include intravascular, intravenous, subcutaneous, intravascular and oral administration.

The agent to be tested may also be administered by gene therapy methods, for example using viral vectors. Such vectors, include retroviral, adenovirus or adeno-associated viral vectors. For a general review of the methods of gene therapy see Strauss, M. and Barranger J. (1997, Concepts in Gene Therapy by Walter de Bruyter & Co., Berlin).

The symptoms and progression of neurodegeneration may be assessed by histopathology or by determining the structure of the animals' neurofilaments, microtubules and axonal diameter, amyloid deposition, or formation of neurofibrillary tangles. For example, after administration of the agent to the animal for a suitable period of time, the treated animals are sacrificed and the NF content in axonal cross sections and axonal calibar are compared to non-treated control genetically non-human animals. A statistically significant decrease in, for example, neurofibrillary tangles, amyloid deposition, or an increase in axonal diameter is indicative of an agent useful for treatment or prevention of neurodegeneration. In addition, tests can be conducted to identify changes in behavioral or motor neuron functions, such as for example development of tremors or muscle weakness, between treated animals and non-treated control animals.

In addition, cell lines derived from the genetically engineered laboratory animals may be used as cell culture models for neurodegenerative disorders. While primary cultures derived from the genetically engineered laboratory animals may be utilized, the generation of continuous cell lines is preferred. Continuous cell lines derived from a transgenic animal may be generated using methods know in the art (See, Small et al., 1985, Mol. Cel. Biol. 5:642-648).

In a specific embodiment of the invention, screening can be carried out by contacting the agent to be tested to cells derived from the genetically engineered laboratory animal. For example, the cells can be exposed to an agent suspected of exhibiting an ability to ameliorate neurodegenerative disorder symptoms, at a sufficient concentration and for a time sufficient to elicit such an amelioration in the exposed cells. After exposure, the cells are examined to determine whether a specific cellular phenotype found to be associated with a neurodegenerative disorder, i.e., has been altered to resemble a phenotype more likely to produce less severe disorder symptoms.

### 6. EXAMPLE

### Targeted Disruption of the NF(H) Gene

In the example presented in this Section, transgenic mice were generated with a targeted disruption of the NF(H) gene. As demonstrated by RNA and protein analysis, the NF(H) protein was not expressed.

### 6.1. Materials and Methods

### 6.1.1. The NF(H) Gene

Isologous genomic DNA for mouse NF(H) was isolated from a 129 Sv/Ev mouse genomic library prepared in λ Dash. Screening was with a probe from a previously isolated mouse genomic clone λ5a (Shneidman, P. S. et al., Mol. Brain Res., 1988, 4:217-231). A random primed Pst I/Not I 430 bp probe (seq −385 to +46) was used for initial screening and positive clones were re-screened with a 54 base oligonucleotide complementary to the initial 5′ coding region of mouse NF(H).

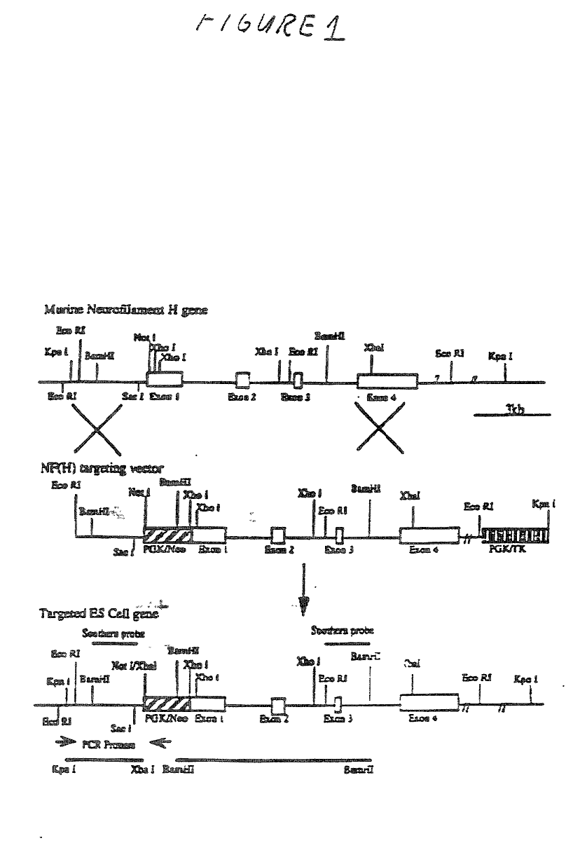

Restriction mapping confirmed that one clone (λNF-H 129) was similar to the known structure of mouse NF(H) and contains the entire coding region plus about 2 kb upstream of the first exon. A map of the relevant portions of the gene and the targeting strategy is shown in FIG. 1.

### 6.1.2. Plasmids and vectors Used

pGEM7 (KJ1)Sal (gift of R. Jaenisch) contains the neomycin resistance gene linked to a phosphoglycerol kinase-1 (PGK-1) promoter (540 bp) and PGK-1 3′ non-translated sequence including the polyadenylation site. pGEM 7 (TK) Sal I (gift of R. Jaenisch) contains the Herpes virus thymidine kinase gene also driven by the PGK-1 promoter and containing the PGK-1 polyadenylation site. pBS/Neo (gift of K. Andrikopoulos) contains the Neo expression vector from pGEM7 (KJ1)Sal cloned into pBluescript (pBS, Stratagene) as an Eco RI/Sal I fragment. Targeting vectors were designed to utilize the positive/negative selection procedure described by Mansour et al. (Nature, 1988, 336:348-352). Two targeting vectors were constructed. Both vectors replaced 370 bp of mouse NF(H) exon 1 with the PGK/Neo resistance gene. One (PΔNF-H 1) contained 2.1 kb of 5′ and 4.2 kb of 3′ homologous sequence. A second vector (PΔNF-H 2,) contained homologous sequence extending 13.4 kb on the 3′ side.

### 6.1.3. Introduction of DNA into ES Cells

Electroporations were initially performed in D3 cells and later in R1 cells. ES cells were split within 24 hr of electroporation. Targeting vector was linearized, extracted with phenol/chloroform, ethanol precipitated and dissolved in sterile H₂O. 50 μg of plasmid DNA was electroporated into 10⁷ ES cells with a BioRad Gene Pulser at 125 μF and 400 V at room temperature. 2×10⁶ cells were plated per 100 mm dish in a nonselective media for 2 days and then selected in 150 μg/ml G418 (Gibco) plus 2 μM ganciclovir (Syntex Research, Palo Alto, Calif.). Neo resistant colonies were isolated after 10-14 days. Colonies were initially picked onto feeder layers of mitomycin C treated mouse embryonic fibroblasts in 96 well microtiter plates. Embryonic fibroblasts were derived from mid-gestation Balb/c mice as described in Robertson (Robertson, E.J., 1987, IRL Press: Washington D.C. P. 71-112). Duplicate 96 well plates were initially established. One plate was frozen as described in Wurst and Joyner (Wurst, W. et al., 1993, IRL Press: New York. p. 33-61) until screening results were known. Clones in the second plate were expanded into 24 well plates without feeder layers and DNA was prepared (Wurst, W. et al., 1993, IRL Press: New York. p. 33-61). Clones were screened by Southern blotting for the pΔNF-H 1 and by PCR using primers as indicated above derived form the Neo gene and flanking 5′ NF(H) sequence for pΔNF-H 2. PCR products were detected by Southern blotting. The “small” NF(H) construct pΔNF-H 1 failed to yield any targeted clones while the “large” NF(H), pΔNF-H 2 yielded one targeted clone in each line at a frequency of approximately 1 in 200. Correct targeting was confirmed by Southern blotting. Suspected targeted clones based on PCR screening were digested with Kpn I, Xba I or Kpn I/Xba I and probed with the Bam HI/Sac I 1300 bp fragment diagramed above. Successful targeting generates a 2.9 kb fragment caused by the introduction of an Xba I site at the 5′ end of the PGK/Neo resistance gene. This fragment was seen in one potentially targeted R1 cell clone.

### 6.1.4. Production of Knockout Chimeric Mice

Chimeras were generated in the Mt. Sinai Transgenic Core Facility essentially as described by Bradley (Bradley, A. 1987, IRL Press: Washington D.C. P. 113-151). Electroporated ES cells were injected into the blastocoele cavity of C57B1/6 blastocysts at day 3.5 post coitum and blastocysts were re-implanted into the uteri of pseudopregnant recipient mice at day 2.5 post coitum. Chimeras were identified on the basis of agouti coat pigmentation. Chimeras were then mated with wild type BALB/c mice and pigmented offspring were tested by Southern analysis of tail DNA. Male chimeras with germline transmission were bred with Swiss white females. Heterozygotes were mated to generate mice homozygous for the targeted mutation.

### 6.1.5. RNA Analysis

Levels of mouse mRNA were determined by RNase protection assays or Northern blotting. Adult mice were sacrificed by cervical dislocation. Tissues were homogenized in 4 M guanidinium thiocyanate with a tissumizer (Tekmar, Cincinnati, Ohio) and the RNA pelleted through step gradients of 2.4 M and 5.7 M CsCl in an SW41 rotor at 30,000 RPM for 24 hr. RNA pellets were solubilized in 10 mM Tris-HCl (pH 7.5), 1 mM EDTA, 5% sodium lauryl sarcosine and 5% phenol and then extracted with phenol/chloroform before being precipitated twice with sodium acetate and ethanol.

RNase protection assays were performed with uniformly labeled RNA probes synthesized with T3 or T7 RNA polymerase and 100 μCi of α-³²P UTP utilizing an RNA transcription kit (Stratagene). The use of mouse NF(L), NF(M), β actin and GAPDH probes in quantitative RNase protection assays have been previously described (Tu, P.-H. et al., J. Cell Biol., 1995, 129:1629-1640; Elder, G. A. et al., Mol. Brain Res., 1992, 15:85-98; Lee, V. M.-Y. Et al., Mol. Brain Res., 1992, 15:76-84). Probes were hybridized to 10-20 μg of total cellular RNA. After overnight hybridization at 45° C., samples were digested with RNase A (80 mg/ml, Sigma) and RNase T1 (700 units/ml, Boehringer-Mannheim) for 1 hr at 30° C. and then digested with proteinase K (125 μg/ml), phenol/chloroform extracted and ethanol precipitated. Protected fragments were run on a 6% non-denaturing gel and localized by autoradiography. For quantitative studies each reaction contained 5 mg brain or cord RNA and 5 mg tRNA. Quantitation was done by densitometry. To ensure linearity a set of RNA standards containing 0-10 μg wild-type brain RNA supplemented with tRNA as needed to give 10 μg in each sample was run in all experiments. Experimental samples were run in duplicate. The levels of β actin or GAPDH expression was used to normalize results.

For Northern blotting 10-20 μg of total cellular RNA was separated in a 1% agarose gel containing 0.66 M formaldehyde. The gel was stained with acridine orange and transferred to Nytran membranes (S&S) according to the directions of the manufacturer. The membrane was prehybridized for 1-2 hr at 42° C. in 50% formamide, 5×Denhardts, 5×SSPE, 0.1% SDS and 100 mg/ml denatured salmon sperm DNA and then hybridized overnight in the same solution with 10⁶ cpm of a 32^{P} labeled random primed probe. Blots were washed to a stringency of 0.1×SSPE/0.1% SDS at 60° C.

### 6.1.6. Western Blotting

Tissue was homogenized using a Heat System Sonicator in lysis buffer containing 50 mM Tris pH 7.4, 100 mM NaCl, 10 mM BDTA, 10 mM EGTA, 1% SDS and 1 mM PMSF, 25 mg/ml leupeptin, 25 mg/ml pepstatin and 50 mg/ml aprotinin. Tissue extracts were analyzed on 10% SDS-polyacrylamide gels, followed by Western blotting with appropriate antibodies. In control experiments, antibody was reabsorbed with 20 mg/ml of the peptide antigen when available.

### 6.1.7. Immunofluorescent Staining

Several anti-neurofilament antibodies are available, including a polyclonal anti-mouse NF(L) (V. Lee), an anti-mouse NF(H) Mab (Boehringer-Marnheim), a mouse MAb RM0108 that recognizes phosphorylated mouse NF(M) (V. Lee) and SMI-32 which recognizes non-phosphorylated mouse and human NF(M) and NF(H) (Sternberger Monoclonals). Control and mutant mice were perfused with buffered 4% paraformaldehyde and the brain, cord, peripheral nerve and root removed and either prepared for frozen sections by immersion in sucrose or post-fixed in paraformaldehyde and stored in PBS for vibratome sections. Cryostat sections were cut at 6-10 μm thickness and vibratome at 50-100 μm and stained as previously described (Elder, G. A. et al., Mol. Brain Res., 1992, 15:85-98; Lee, V. M.-Y. et al., Mol. Brain Res., 1992, 15:76-84; Elger, G. A. et al., J. Neurosci. Res., 1996. In press.). Tissues from fetal or early postnatal animals were immersion fixed in buffered 4% paraformaldehyde. Single or double immunofluorescence staining was performed with the antibodies listed above and visualized with species specific fluoresceinated or biotinylated secondary antibodies (Amersham) and fluorescein or Texas red labeled streptavidin. Most sections were counter-stained with the blue-emitting fluorescent dye 4′,6-diamidino-2-phenylindode hydrochloride (DAPI). Sections were mounted in 90% glycerol+DABCO and analyzed and photographed using a Leica laser scanning confocal microscope or a Zeiss Axiophot microscope.

### 6.2. Results

Multiple attempts to generate chimeric animals with the D3 derived clone failed. However, injections of the R1 derived clone into C57Bl/6J blastocysts yielded one strongly chimeric male identified on the basis of agouti coat pigmentation. Chimeric animals were bred to generate mice homozygous for the mutation of interest.

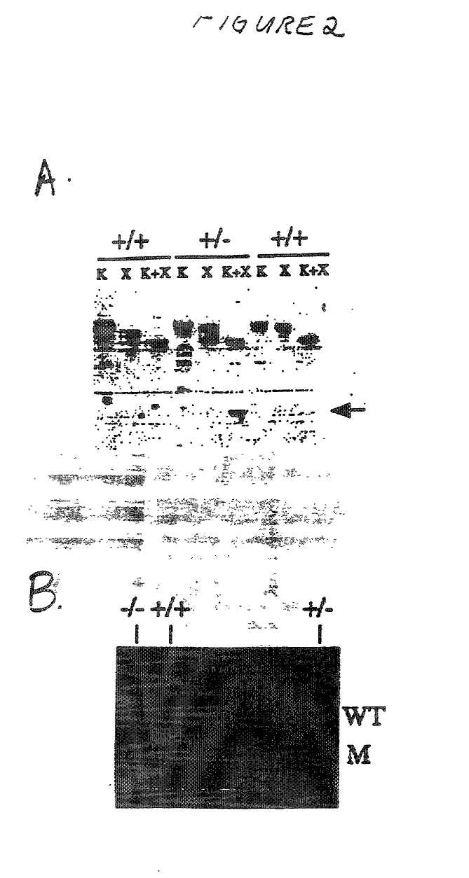

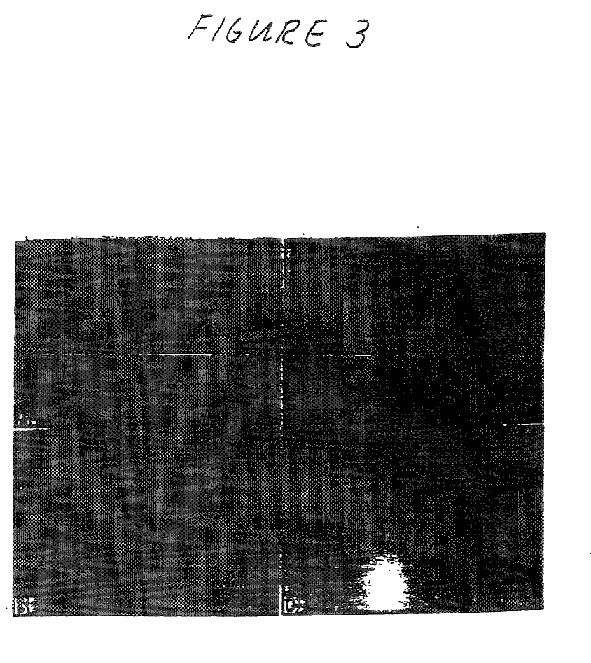

In FIG. 2A, a Southern blot is shown of a targeted ES cell clone digested with Kpn I (K), Xba I (X), or a Kpn I/XbaI double digest and probed with the Bam HI/Sac I fragment indicated above. Two clones show the expected wild type pattern (+/+) while a targeted clone (+/−) gives an additional Xba band and a 2.9 kb Kpn/Xba band (indicated by arrow) consistent with homologous recombination. In FIG. 2B a Southern blot of offspring from a heterozygous/heterozygous mating is shown. DNA was digested with Bam HI and probed with the Bam HUEco RI fragment shown above. Several homozygous (−/−) animals containing only the recombinant band are present.

This chimeric male was mated with C57Bl/6J females and the mutated gene was successfully transmitted to approximately 50% of offspring. In subsequent matings of these offspring the targeted NF(H) gene was transmitted as expected in a Mendelian manner and was bred onto a Swiss outbred background and onto the 129 Sv/Ev background.

In order to verify that gene inactivation had occurred wild type, heterozygous and homozygous mice were analyzed for expression of NF(H) protein by Coomassie Blue staining of protein gels and by Western blotting with antibodies to NF(H), NF(M) and NF(L). No NF(H) protein could be detected on Coomassie Blue stained gels even though the normal NF(H) band is easily identifiable on crude extracts of mouse brain and spinal cord. Western blotting with a polyclonal antibody to the last 20 amino acids of human NF(H) showed no NF(H) band in the homozygous mutant animals even thought readily detectable amounts of NF(H) were present in heterozygous and wild type mice. Also immunocytochemical staining of mouse brain sections (FIG. 3) revealed no NF(H) staining in homozygous mutants even though readily detectable amounts of NF(L) could be seen in the same double labeled sections and strong staining could be seen with the NF(H) antibody in control wild type mice.

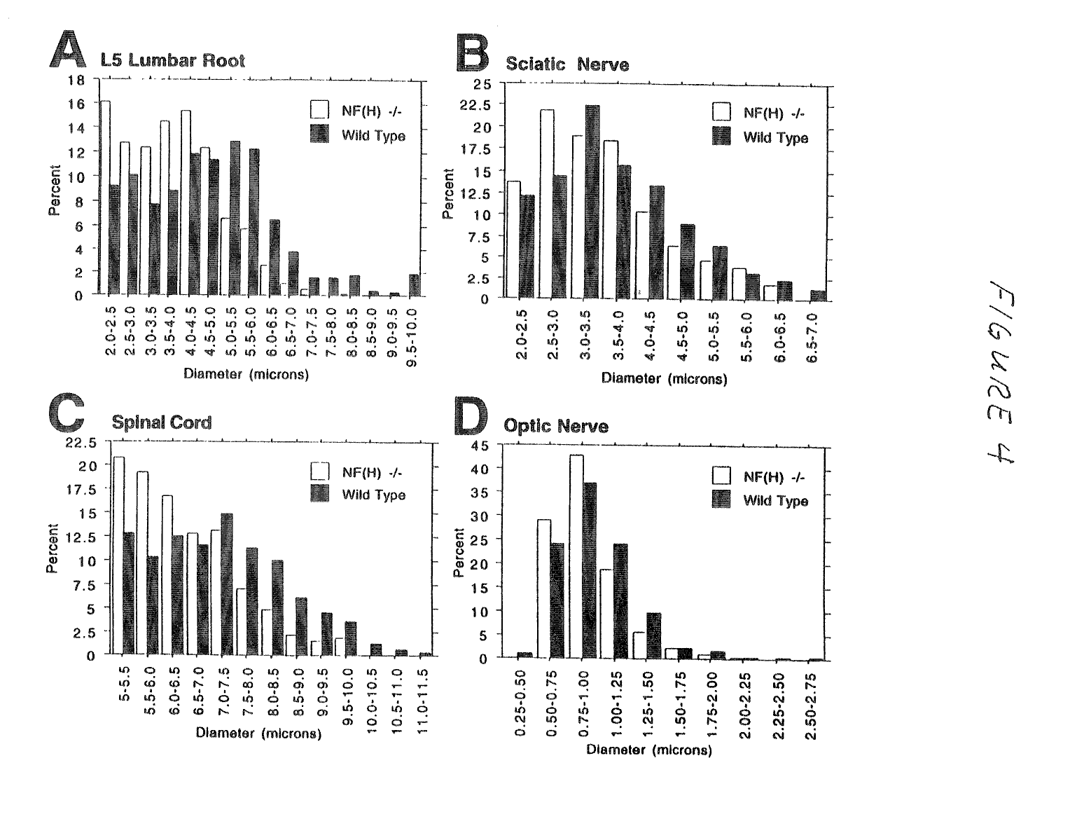

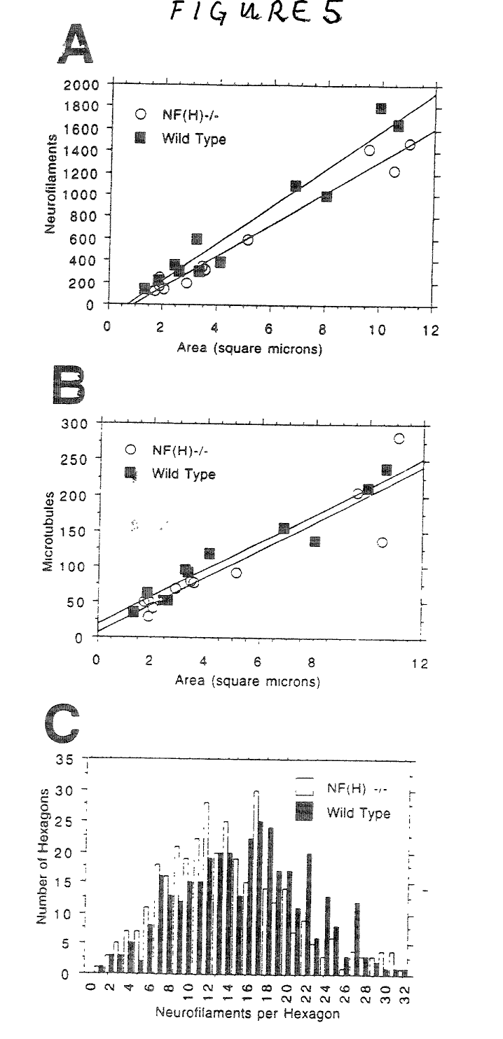

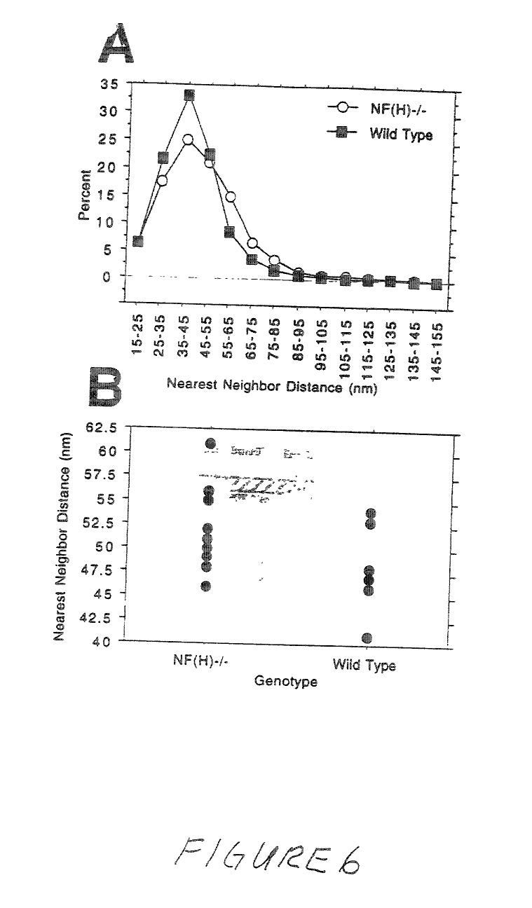

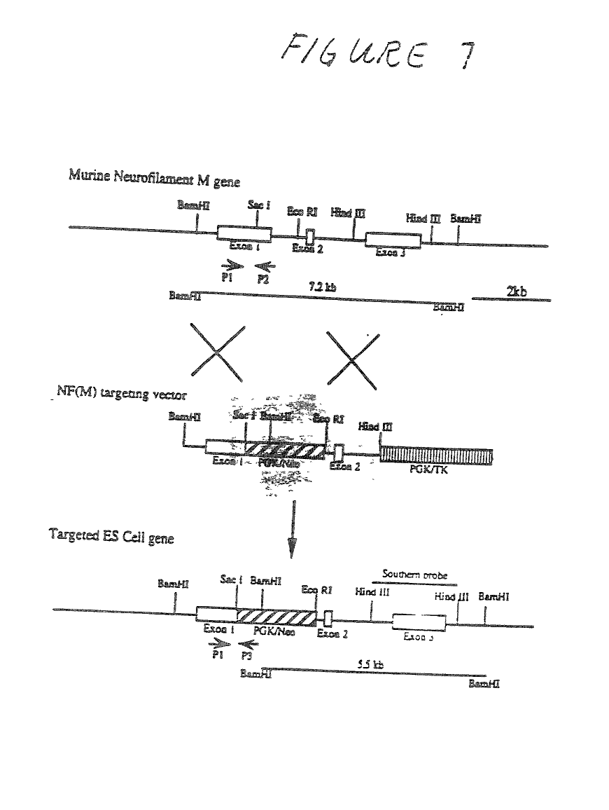

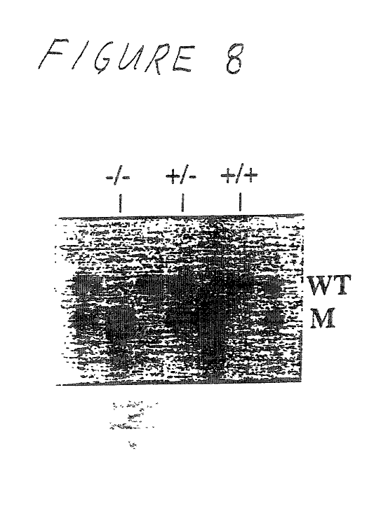

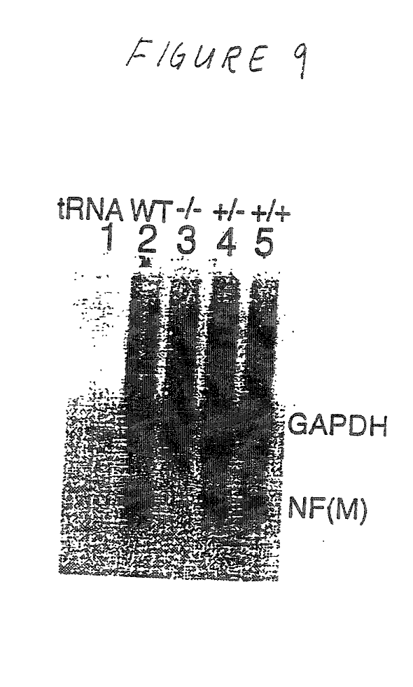

Examination of the frequency distribution of axonal diameters in control and mutant roots (FIG. 4A) revealed that over 40% of myelinated axons in wild type roots were larger than 5.0 μm compared to only 16.6% in the null mutant (p=0.0008, unpaired t-test). Only rare axons in the mutant reached diameters greater than 6.5 μm even though over 9% were in this class in wild type roots. The loss of large diameter myelinated axons was accompanied by a shift towards medium and smaller diameter fibers.

Similar changes were seen in a mophometric analysis of axonal diameters in sciatic nerves from NF-H mutant and control animals (FIG. 4B). Average diameters in the proximal segment of the nerve decreased from 3.6+/−1.0 (S.D.) μm in wild type to 3.4+/−1.1 mm in NF-H null mutants (p=0.0145, Mann-Whitney U test). The frequency distribution of axon diameters in sciatic nerves again revealed a shift towards small and medium size axons.

In the CNS axon diameters were also effected. In the ventral medial portion of the cervical cord segment (a region containing many large axons) the diameters of axons over 5 μm in size were decreased from 6.13+/−1.91 μm in control to 5.8+/−1.59 (p>0.0001, Mann-Whitney U test) (FIG. 4C). Likewise in the optic nerves (an area of the CNS containing relatively small myelinated axons nearly all less than 2 μm in diameter) average axonal diameters decreased from 0.97+/−0.30 μm in wild type to 0.91+/−0.26 μm in NF-H null mutants (p=0.0004, Mann-Whitney U test. In both regions the frequency distribution of axonal diameters showed a shift towards small diameter fibers in the mutant (FIG. 4D). Thus, the presence of the NF-H subunit is required to achieve maximal axon diameter in all size classes of myelinated axons in both CNS and PNS.

To determine if NF content was altered in the null mutants, NFs were counted in both myelinated axons of a range of sizes and NF numbers were plotted against axonal area. As shown in FIG. 5 myelinated axons in the null mutant appeared to contain slightly fewer NFs than comparably sized axons in controls although microtubule numbers were unchanged.

To measure NF densities more directly in these same axons we used methods similar to those described by Price et al., (1988, J. Neurocytol. 17:55-62). NFs densities were determined by applying a template of hexagons )every hexagon equivalent to an area of 0.10 sq. microns) over individual electron micrographs and counting the number of NFs in each hexagon. A frequency distribution plot was generated showing the number of NFs per hexagon (FIG. 6A). The average number of NFs per hexagon was reduced from 15.7+/−6.3 (S.D.) In control axons to 14.1+/−6.3 in the mutant (p=0.0005, unpaired t-test) and as shown in FIG. 6A the frequency distribution was shifted in the mutant towards hexagons containing fewer Nfs, demonstrating that NFs are slightly less densely packed in the NF-H mutant supporting the conclusion that axons in the NF-H −/− animals contain approximately 10% fewer NFs.

### 7. EXAMPLE

### Generation of NF(M) Knockout Mice

Using methods similar to those set forth for the production of NF(H) knock-out mice, NF(M) knock-out mice were produced.

### 7.1. Materials and Methods

### 7.1.1 Generation of NF(M) Knockout Mice

A murine NF(M) clone was isolated from the 129 Sv/EV genomic library described above with a 1600 bp Eco RI/Hind III probe containing the third exon of mouse NF(M) (Levy, E., et al., Eur. J. Biochem., 1987, 166:71-77). An 11 kb clone was isolated. A targeting strategy similar to that utilized for NF(H) was followed using a targeting vector containing 2 kb of 5′ and 1.5 kb of 3′ homologous sequence (FIG. 4). Methods for generating the NF(M) mice where as described above. In addition two larger constructs containing extended 3′, or 5′ and 3′ sequence were constructed. Successful targeting was obtained with the initial construct in the R1 ES cell line (targeting frequency approx 1/150). One clone was introduced into mouse blastocysts and three chimeric offspring were obtained. One female chimera died before successfully breeding while two male chimeras produced agouti offspring containing the mutated NF(M) gene. Offspring from these and subsequent matings were bred to homozygosity. RNase protection assay were performed as described above.

### 7.2. Quantitative Western Blot Analysis

Quantitative Western blots were performed as previously described with minor modifications (Tu, P.-H., et al., J. Cell Biol., 1995, 129:1629-1640). In brief, tissue was homogenized, sonicated in BUST buffer (50 mM Tris-HCL, pH 7.4, 8 M urea, 2% β-mercaptoethanol and 0.5% SDS), and centrifuged at 40×10³ rpm, at 25° C. for 30 min in a TL-100 ultracentrifuge (Beckman Instruments, Inc., Fullerton, Calif.). Protein concentration in the supernatants were determined using the Coo-massie protein assay method (Pierce Chemical Co., Rockford, Ill.) according to the manufacture's instructions. Each sample was loaded in triplicate and each lane contained 40 μg of total protein from neocortex or hippocampus or 10 μg of total protein from spinal cord, brainstem, or sciatic nerve. Blots were cut into three parts. The top third was incubated overnight with RMO24 for detection of NFHP+++ level or RMdO9 for NFIP−−− level; the middle third was incubated with RMO189 for total NFM level, or RMO55 for NFMP+++; and the lower third, was incubated with a rabbit anti-NFL polyclonal antiserum for total NFL, or a mouse mAb anti-β-tubulin (Amersham Pharmacia Biotech Inc., Piscataway, N.J.) for tubulin levels. Each part was then incubated for 1 h with 10 μCi¹²⁵I-conjugated goat anit-mouse IgG for the mouse mAbs (RMO24, RMO55, RMO189, and anti-β-tubulin) or ¹²⁵I-conjugated Protein A for the rabbit anti-NFL polyclonal antisera. The dried blots were exposed to Phosphor-Imager plates for various time periods and individual bands were visualized and quantified with Image Quant software (Molecular Dynamics, Inc., Sunnyvale, Calif.). Western blotting to detect NH₂-terminal epitopes of NF-M was performed with a polyclonal antiserum raised against the NF-M head domain (1994, Manipulating the Mouse Embryo: A Laboratory Manual, 2^{nd} edition).

### 7.3. Electron Microscopy

Mice were anesthetized and fixed by vascular perfusion with a solution containing 2% formaldehyde (from paraformaldehyde), 1% glutaraldehyde, and 0.12 M sodium phosphate buffer, pH 7.4. The brain, spinal cord, optic nerves, sciatic nerve, and L5 lumbar roots were dissected out, postfixed in buffered osmium tetroxide, and embedded in Epon by routine methods. Thin sections were examined using an JEOL 100CX electron microscope.

To count neurofilaments and microtubules, cross sections of axons were photographed at a magnification of 20,000 and then enlarged an additional two and one-half fold during printing. NF densities were determined using methods similar to those described by Price et al.(1988, J. Neurocytol. 17:55-62) by laying a template of hexagons on each print. Hexagons had 11-mm sides, equivalent to an actual print area of 0.10 μm². NFs in all hexagons that fell completely within axonal borders were counted. Hexagons were excluded only if vesicular organelles filled more than ˜10% of the hexagon. Nearest neighbor distances were computed from the x/y coordinates of NFs in representative electron micrographic prints.

### 7.4. Measurement of Axonal Diameters

For measuring axonal diameters, 1-μm thick transverse sections of L5 ventral root, sciatic nerve, or spinal cord were stained with toluidine blue and photographed through a Zeiss Axiophot microscope with a 10× or 20× objective 2×2 slide images were scanned into the program Adobe Photoshop 3.05 using a Kodak 35 mm rapid film scanner. Images were enlarged three- to four-fold and printed. Optimal brightness and gray scale pixel values were adjusted to as to provide the sharpest discrimination of the myelin/axon border. Axon profiles were traced in nonoverlapping contiguous fields using a digitizing tablet. The area of myelinated axons was then measured using the program NIH-Image and axons were assumed to be circular for purposes of diameter calculations. In measuring the sciatic nerves, all myelinated axons in the largest trunk of the nerve were numbered and every fifth axon was sampled chosen by a set of random numbers. In optic nerve, random fields were photographed in the electron microscope at a magnification of 4,800 and then enlarged an additional two and one-half fold during printing. Every third myelinated axon was sampled in five randomly chosen fields. Statistical analysis (unpaired t test or Mann-Whitney U test) was performed using the program StatView (Abacus Concepts, Inc., Berkeley, Calif.).

### 7.2. Results

### 7.2.1. Production of Mice Bearing a Null Mutation in the NF-M Subunit

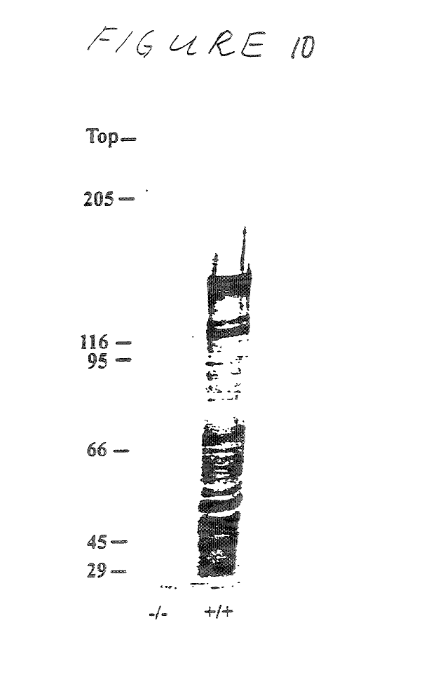

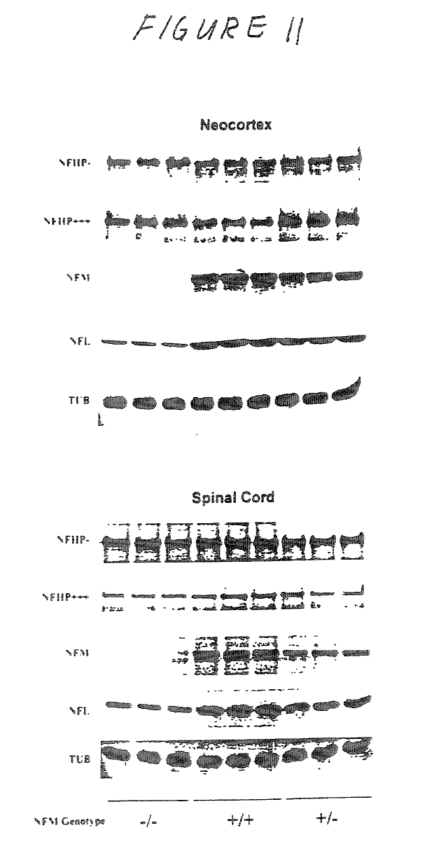

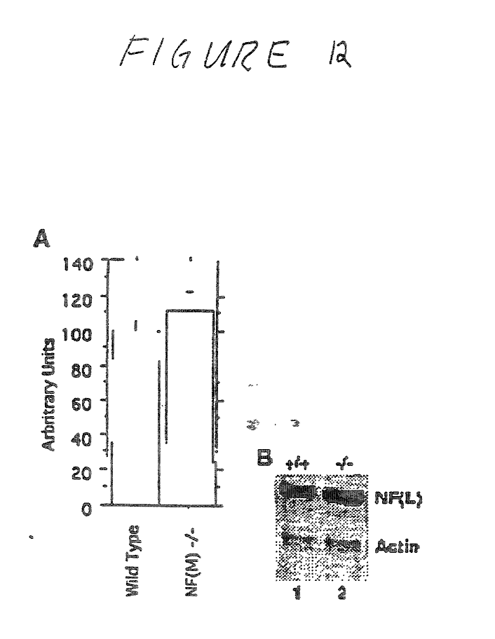

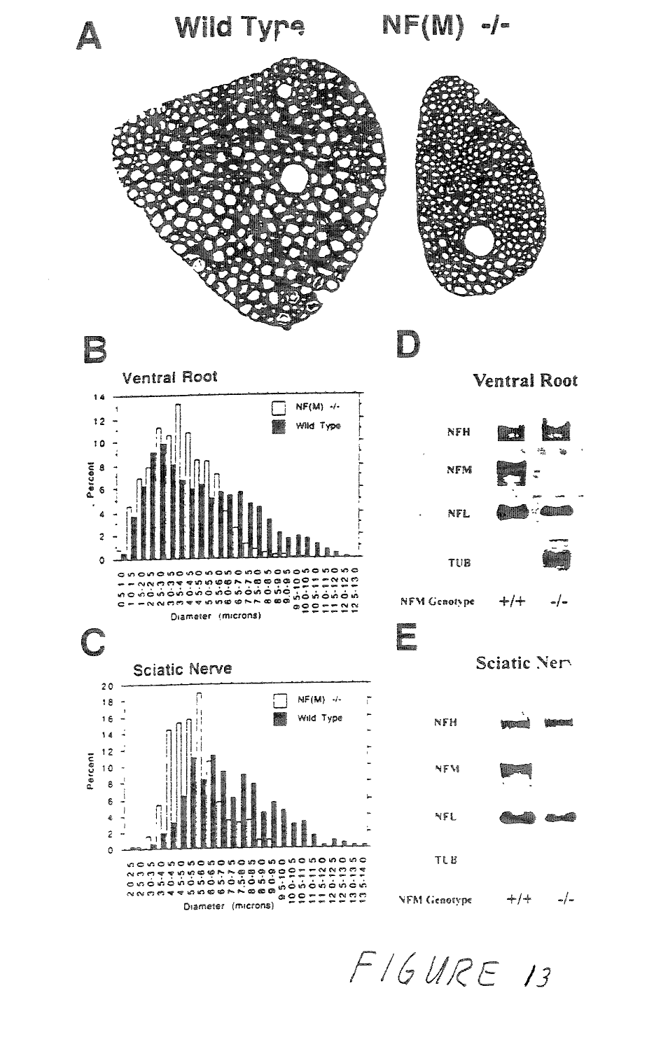

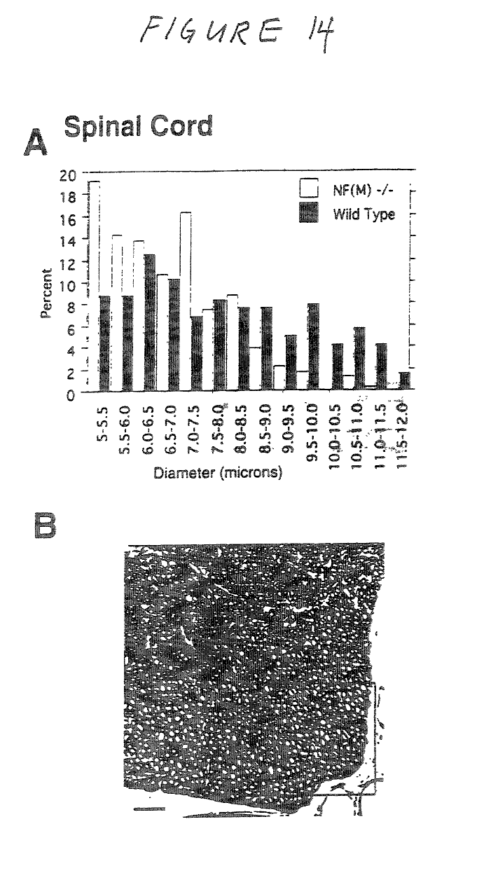

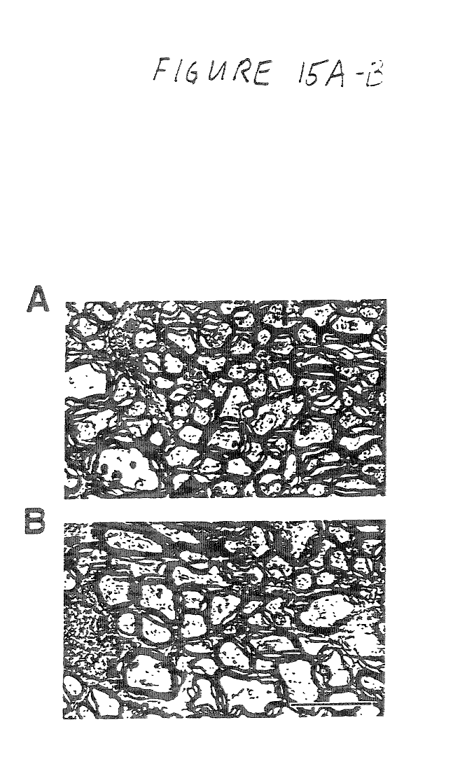

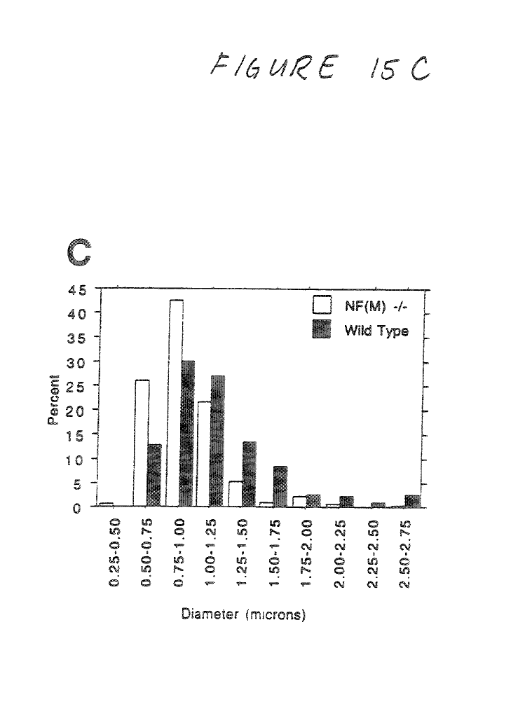

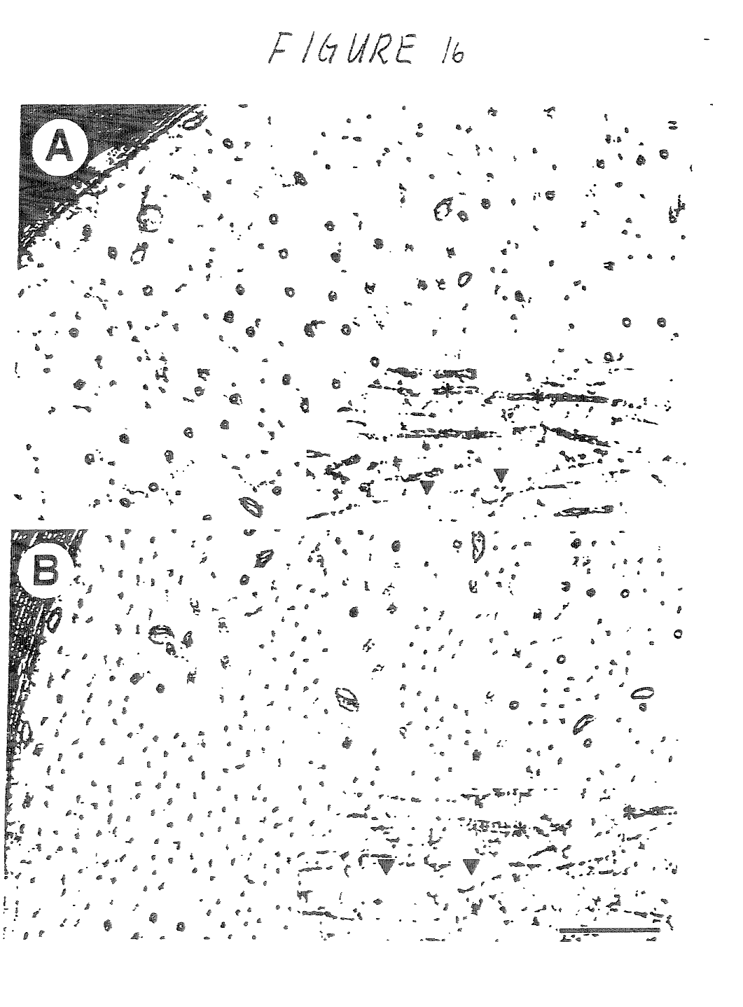

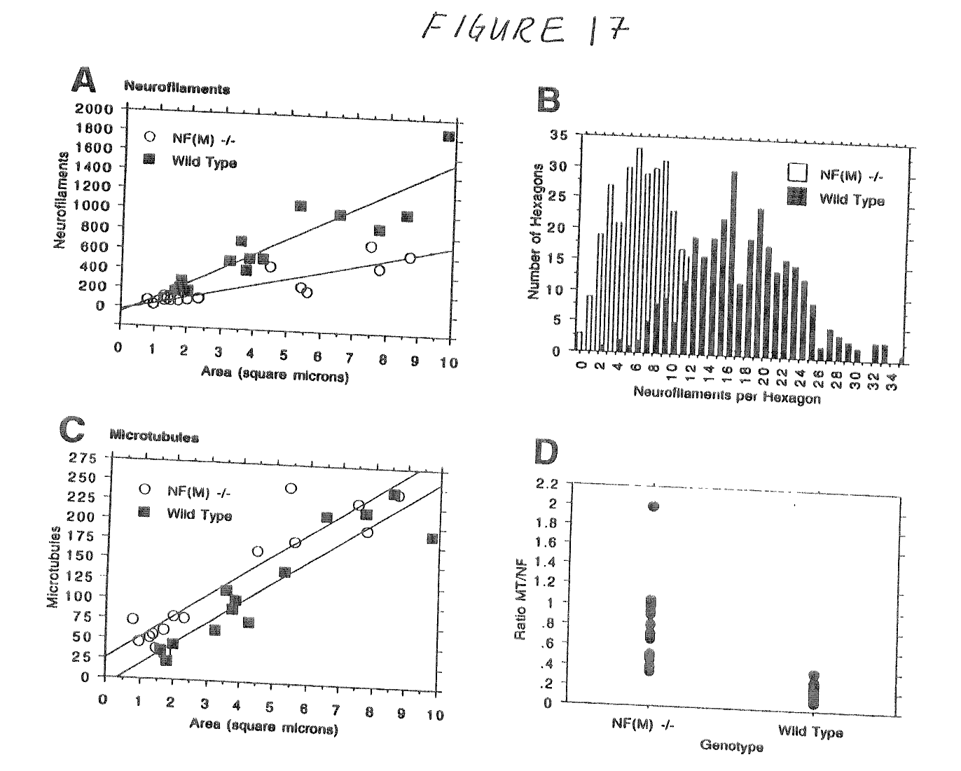

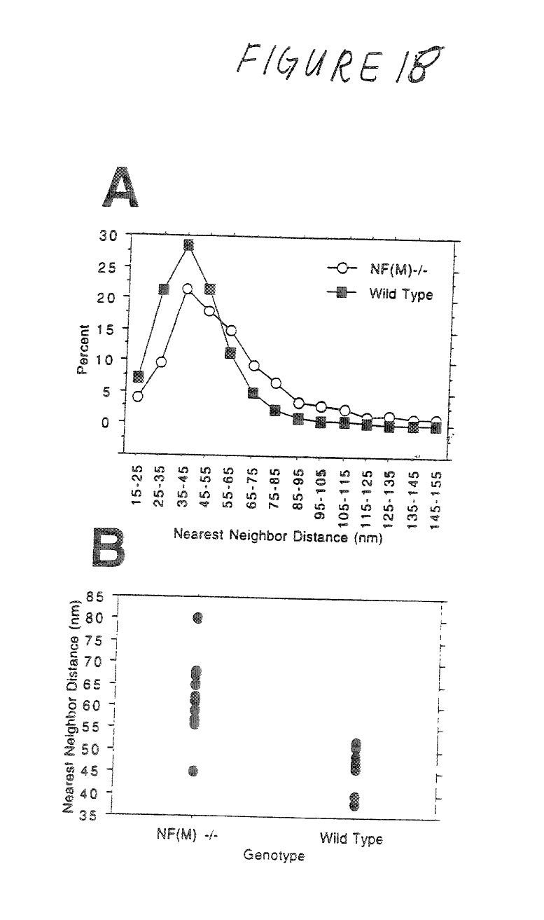

The targeting strategy for generating NF-M null mutant mice is illustrated in FIG. 7. Lines were established by breeding a male chimera with C57BL/6 females. Male heterozygotes from these matings were subsequently bred with 129 Sv/J or Swiss-Webster females. On all genetic backgrounds the mutant allele was transmitted in a Mendelian fashion. We have studied animals both on a mixed 129/C57BL background and 129/C57BL hybrids bred to outbred Swiss-Webster mice. We have not detected any qualitative effects of genetic background on the phenotype of NF-M null mutation.

RNase protection assays with probes either 5′ (exon 1) or 3′ (exon 3) to the neomycin resistance gene revealed that no NF-M MRNA could be detected in the NF-M homozygous null mutants (FIG. 9). As shown in FIG. 10, Western blotting confirmed the absence of NF-M protein in the null mutant. We were also unable to detect NF-M protein in sections of brain from NF-M homozygous animals stained immunocytochemically with monoclonal anti-NF-M antibodies even though in sections double labeled with a polyclonal andi NF-L antisera, NF-L staining was present.

### 7.2.2 NF-L Levels are Decreased and NF-H Levels are Increased in the NF-M Null Mutant

Since rodent NFs are obligate heteropolymers and are assembled into filaments with a defined stoichiometry in vivo, we determined how the lack of NF-M expression would affect levels of the other NF subunits by Western blotting. Representative Western blots of neocortex and spinal cord of NF-M null, heterozygous, and control mice are shown in FIG. 11. Quantitations showed that the immunoreactivities of both the phosphorylation-independent (NEMPi) and highly phosphorylation dependent (NFMP+++) epitopes of NF-M decreased in the neocortices of heterozygous mice and were undetectable in the NF-M null mice. Both NFMPi and NFMP+++ decreased in the heterozygous mice by ˜40%. Thus, the decreases in NF-M immunoreactivities were not affected by the phosphorylation state of NF-M suggesting that these decreases result from reduced levels of total NF-M rather than a change in the phosphorylation state.

Interestingly, a concomitant decrease in the level of NF-L was also detected in the NF-M heterozygous and NF-M null mice. Quantitations showed that the extent of decrease in NF-L levels were comparable to those of NF-M (FIG. 11). For example in neocortex, the level of NF-L decreased in NF-M heterozygous mice by ˜50%, similar to the decrease in NF-M. Furthermore, in neocortex, the NF-M null mice that lacked all NF-M protein contained only 13% of the level of NF-L found in control mice. Dramatic decreases in NF-L were also observed in other regions including spinal cord (FIG. 11), hippocampus, brainstem, and cerebellum. These data are consistent with our previous observations that NF-M levels regulate the level of NF-L protein to maintain the relative stoichiometry between NF subunits (Tu, P.-H. Et al., J. Cell Biol., 1995, 129:1629-1640).

By contrast, immunoreactivities of antibodies to the heavily phosphorylated (NFHP+++) and nonphosphorylated (NFHP−−−) epitopes of NF-H increased in the neocortices of both NF-M heterozygous and null mice (FIG. 11). Quantitations showed that the levels of NF-H in neocortex increased 20-50% in both the NF-M heterozygous and NF-M null mice. An increase in the level of NF-M has been shown to down-regulate the phosphorylation state of NF-H by ˜20% (Tu, P.-H. et al., J. Cell Biol., 1995, 129:1629-1640). Thus, the increases in NF-H levels in the NF-M mutants are likely to be due, at least in part, to the changes in NF-M levels although a secondary effect of altered NF-L levels on NF-H is also possible. Interestingly, unlike neocortex, NF-H was not increased in spinal cord (FIG. 11). This is consistent with our previous observation that overexpression of human NF-M in transgenic mice does not affect NF-H levels in spinal cord (Tu, P.-H. et al., J. Cell Biol., 1995, 129:1629-1640). Taken together, these data clearly demonstrate that expression of NF-L and NF-M is coordinately regulated in many CNS regions and that levels of NF-M and NF-H are coregulated in an inverse manner in neocortex but not in spinal cord. As shown in FIG. 11, the level of β-tubulin in brain and spinal cord was not affected by the changes in NF subunits in the NF-M heterozygous and null mice, remaining comparable to that of control mice.

Previously, when human NF-M was overexpressed in transgenic mice, the change in NF-L protein level appeared to reflect changes in posttranscriptional regulation since no change in NF-L mRNA level occurred (Tu, P.-H. et al., J. Cell Biol., 1995, 129:1629-1640). To quantitate NF-L MRNA levels in the NF-M null animals we performed quantitative RNase protection assays as previously described (Tu, P.-H. et al., J. Cell Biol., 1995, 129:1629-1640). As in the overexpression experiments reported earlier, MRNA levels for NF-L were unchanged in the null mutant (FIG. 12) implying that NF-L protein levels are being regulated posttranscriptionally.

### 7.2.3. Diminished Axonal Diameters in NF(M) Null Mutant Mice

A role for NFs in establishing axonal diameter has long been suspected from the correlation between NF content in axonal cross sections and axonal caliber (Hoffman, P. N., 1984, J. Cell Biol. 101:1332-1340). This view has been reinforced by several recent animal models that have shown that radial growth of myelinated axons is severely inihibited in axons lacking all NFs (Eyer et al., 1994, Neuron 12:389-405; Ohara,O., 1993, J.Cell. Biol. 121:387-395; Yamasaki, H., 1991, Acta. Neuropathol. 82:427-434; Zhu, Q. S. et al., 1997, Exp. Neurol. 148:299-316). To examine the effects of the NF-M null mutation on axonal development, we measured axon sizes in PNS (L5 ventral root and sciatic nerve) and CNS (spinal cord and optic nerve) structures.

Toluidine blue-stained sections of L5 ventral roots from a 4-mo-old control and null mutant are shown in FIG. 13A. Myelinated axons appeared generally smaller in the mutant roots with the largest diameter fibers in the mutant failing to reach a caliber similar to the largest axons in control. Morphometric analysis, measuring the area of every myelinated axon within the L5 ventral root confirmed this initial impression (FIG. 13B). Average axonal diameter was decreased from 4.9±2.5 (SD) μm in wild type to 3.9±1.6 μm in the NF-M mutant (P<0.0001, Mann-Whitney U test). Examination of the frequency distribution of axonal diameters in control and mutant roots (FIG. 13B) revealed that >28% of myelinated axons in wild-type roots were >6.5 μm compared with only 6.4% in the null mutant (P=0.028, unpaired t test). Only rare axons in the mutant reached diameters >8 μm event though >9.0% fell into this class in wild-type roots. The loss of large diameter myelinated axons was accompanied by a shift towards medium and small diameter fibers. Although the number of myelinated axons was slightly reduced from 697±42 in wild-type animals to 630±66 in NF-M null mutants, the decrease was not statistically significant (P=0.4111 unpaired t test). Thus, the decreased size of the mutant L5 root appears to be primarily the result of a general reduction in size of all myelinated axons.

Equally dramatic changes were seen in a morphometric analysis of axonal diameters in sciatic nerves from NF-M mutant and control animals (FIG. 13C). Average diameters in the proximal segment of the nerve decreased from 7.2±2.2 (SD) μm in wild type to 5.5±1.2 μm in NF-M null mutants (P<0.0001, Mann-Whitney U test). As in the L5 roots, axonal diameters in sciatic nerve were shifted towards small and medium sized axons and no axons >9.0 μm in diameter were found in the null mutant.

As shown in FIG. 13D and E, neither ventral root nor sciatic nerve contained detectable amounts of NF-M protein, whereas levels of NF-L were reduced by ˜50% and levels of tubulin appeared increased. The latter observation is consistent with the relatively increased numbers of microtubules found in axons in the L5 roots (see below).

To determine whether CNS axons were also effected we examined axon sizes in the spinal cord. We measured all axons>5 μm in diameter in a 1.9×10⁵ μm² area of the ventral medial portion of the third cervical cord segment (FIG. 14B). This region was chosen since comparable areas could be easily identified in different animals and because this region contains many large axons. As shown in FIG. 14A, axons in this region were significantly smaller in the null mutant than in control (P<0.0001, Mann-Whitney U test). Lost large diameter axons (>8 μm) appeared to have been replaced by medium diameter fibers (5-8 μm) in the NF-M null mutant.

Thus, the NF-M null mutation appears to reduce the diameter of myelinated axons in both PNS and CNS. However, since the regions examined (L5 ventral root, sciatic nerve, spinal cord) contain mainly medium and large sized axons it was less clear if the mutation was effecting all sizes of myelinated axons. To determine the effects of the mutation on smaller caliber axons we measured axon sizes in the optic nerves, an area of the CNS containing relatively small myelinated axons nearly all <2 μm in diameter. In electron micrographs of optic nerve, myelinated axons were visibly smaller in the NF-M null mutant than control animals (FIG. 15A and B). Average diameters decreased from 1.16±0.42 μm in wild type to 0.93±0.29 μm in NF-M null mutants (P<0.0001, Mann-Whitney U test). As in the other regions examined axonal diameters were shifted towards smaller diameter fibers in the mutant (FIG. 15C). Thus, the NF-M subunit appears to be required to achieve maximal axonal diameter in all size classes of myelinated axons in both the CNS and PNS.

### 7.2.4. Reduced Neurofilament Content in Mice Lacking an NF(M) Subunit

It remains unclear how NFs contribute to the specification of axonal diameter. One popular idea for the role of the larger NF subunits has been that the phosphorylated tail domains of NF-M and NF-H determine interfilament distance and that spacing between filaments in turn specifies axonal diameter (Carden, M. J. et al., 1987, J. Neurosci. 7:3489-3504; Matus,A., 1988, Trends Neurosci. 11:291-292). If so, then loss of NF-M from NF sidearms should result in more tightly packed NFs and the NF-M null mutant should require more NFs than wild type to produce an axon on equivalent diameter.

To look for an ultrastructural basis for the diminution of axonal diameters in the null mutant we examined electron micrographs of L5 ventral roots from mutant and control animals. NFs were readily apparent in both the null mutant and control (FIG. 16). However, filaments in the mutant animal appeared to be reduced in number although otherwise of normal configuration in both transverse and longitudinal sections (FIG. 16). Microtubules also appeared to be normal in appearance, although in many axons their numbers seemed to be increased.

To determine if NF content was actually altered in the null mutants, NFs were counted in the intemodal regions of axons over a range of sizes and NF counts were plotted against axonal area. As shown in FIG. 17A, axons in the null mutant consistently contained fewer NFs than comparably sized axons in controls. By contrast, these same axons contained more microtubules (FIG. 17C and D) increasing the average ratio of microtubules to NFs from 0.22±0.8 (SD) in wild type to 0.83±0.41 in the mutant axons (P<0.0001, Mann-Whitney U test).

Since axons in the null mutant contain fewer NFs, NF densities in the mutant should also be decreased. We measured NF densities in mutant and wild-type axons using methods similar to those described by Price et al., (1988, J. Neurocytol. 17:55-62). NFs densities were determined by applying a template of hexagons (every hexagon equivalent to an area of 0.10 μm²) over each electron micrograph and counting the number of NFs in each hexagon. A frequency distribution plot was then generated showing the number of NFs per hexagon (FIG. 17B). The average number of NFs per hexagon was reduced from 17.4±5.8 (SD) in control axons to 7.5±4.1 in the mutant (P<0.0001, unpaired t test) and as shown in FIG. 17C the frequency distribution was dramatically shifted in the mutant towards hexagons containing fewer NFs, demonstrating that NFs are less densely packed in the NF-M mutant.

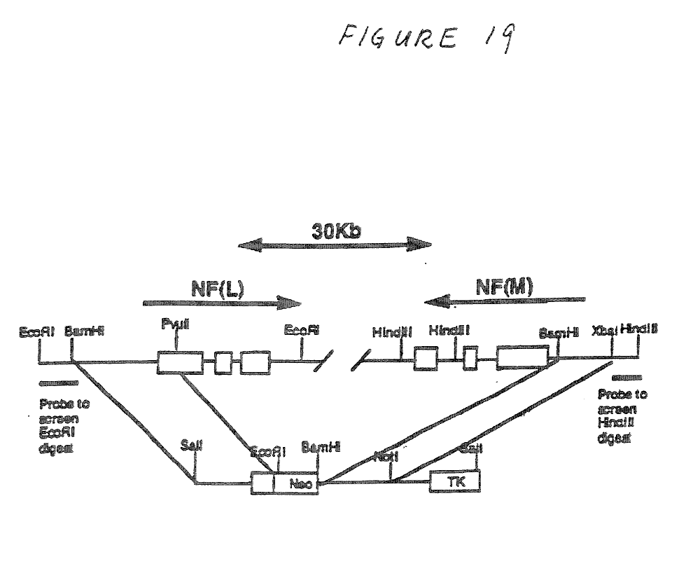

To determine the effect of the reduced NF density on interfilament spacing in the mutant we measured nearest neighbor distances in these same axons (FIG. 18). Mean interfilament distances increased from 46±17 (SD) nm in control to 62±33 in the mutant (P<0.0001, Mann-Whitney U test) although the modal interfilament distance was identical between mutant and control (47 nm). Indeed an analysis of those filaments with nearest neighbors of 60 nm or less revealed that interfilament spacing in the mutant (43±10) was little changed from control (41±10, P<0.0001). Thus, although average interfilament distances were increased (as would be expected due to the decreased filament number) when filaments are closely spaced in the mutant they assume an interfilament distance that is similar to wild type.

Taken together these data are not consistent with the prediction that axons in mutant animals would require more NFs, packed at a closer density in order to produce axons of comparable size to wild type. However, these findings are consistent with suggestions that levels of NF-L determine the number of NFs (Monteiro, M. J., 1990, J. Cell. Biol. 111:1543-1557; Nixon, R. A., 1993, Brain Pathol. 3:29-38).

### 7.2.5 Lack of Overt Phenotype or Major Structural Defects in Mice Carrying a Disrupted NF(M) Gene

No overt phenotype was associated with a null mutation in the NF-M gene. Animals appeared normal at birth and were indistinguishable from littermates. 4-mo-old mutant animals appeared the same size as littermates. Both male and female homozygous mutants were fertile. No striking behavioral changes were apparent and null mutants appeared to have normal motor strength and coordination. NF-M^{-/-} animals have shown no obvious health problems up to one year of age.

A comparison by light microscopy of coronal sections of the brain from 4-mo-old mutant and wild-type animals revealed no obvious differences between null mutants and wild-type animals. Both cortical and subcortical structures appeared normal. In particular large neurons such as the Purkinje cells of the cerebellum and anterior horn cells in the spinal cord that contain large numbers of NFs appeared to have developed normally.

### 8. EXAMPLE

### Production of NF(L) and NF(M) Double Knockout Animals

Since the mouse NF(L) and NF(M) genes are located on the same chromosome within 30 kb of one another, the double mutant L-/M- cannot be easily created by crossing the existing L-line with the M-line. Consequently, it may be desirable to transfect embryonic stem cells with a targeting vector designed to simultaneously inactivate both genes by creating a large deletion that removes elements of both genes. ES cells containing L-/M- deletions may be used to generate chimeric mice that when bred to homozygocity lack functional NF(L) and NF(M) genes. Such mice could be mated with NF(H) null mutants to produce triple knockout mice.

A cosmid clone (IC3) (Campion, D. et al., Human Molecular Genetics, 1995, 4:2737-2377) was kindly provided by Dr. Jean-Pierre Julien. This cosmid contains both the mouse NF(L) and NF(M) genes, approximately 30 kb apart. Restriction mapping of this clone determined that the NF(L) and NF() genes are in opposite orientations. A targeting vector to create null mutations in both genes was constructed by cloning in homologous sequence from the flanking regions of both genes as diagramed in FIG. 19. This vector contains 6 kb of sequence upstream of NF(L) and 3 kb flanking NF(M). ES cell clones can be screened for a homologous recombination event using external probes and digests.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

Various publications are cited herein, the contents of which are hereby incorporated by reference in their entireties.

1. A genetically engineered non-human animal whose cells lack one or more functionally active neurofilament genes.

2. The genetically engineered non-human animal of claim 1, wherein the neurofilament gene is the neurofilament (H) gene.

3. The genetically engineered non-human animal of claim 1, wherein the neurofilament gene is the neurofilament (M) gene.

4. The genetically engineered non-human animal of claim 1, wherein the neurofilament gene is the neurofilament (L) gene and (M) gene.

5. The genetically engineered non-human animal of claim 1, wherein the animal lacks a functionally active neurofilament (H), (M), or (L) gene.

6. The genetically engineered non-human animal of claim 1, wherein said animal is a mouse.

7. A genetically engineered non-human animal expressing one or more human neurofilament genes.

8. The genetically engineered non-human animal of claim 1, wherein said animal expresses one or more human neurofilament genes.

9. The genetically engineered non-human animal of claim 7, wherein the human neurofilament genes are wild type neurofilament genes.

10. The genetically engineered non-human animal of claim 7, wherein the human neuro filament genes encode a mutant human neurofilament gene.

11. The genetically engineered non-human animal of claim 7, wherein the human neurofilament gene is the neurofilament (H) gene.

12. The genetically engineered non-human animal of claim 7, wherein the neurofilament gene is the neurofilament (M) gene.

13. The genetically engineered non-human animal of claim 7, wherein the neurofilament gene is the neurofilament (L) gene.

14. The genetically engineered non-human animal of claim 7, wherein the neurofilament genes are selected from the group consisting of the human neurofilament (H), (M) or (L) genes.

15. The genetically engineered non-human animals of claim 7 wherein the animal is a mouse.

16. A method of making a genetically engineered non-human animal lacking one or more neurofilament genes comprising introducing an altered neurofilament gene that fails to encode a functionally active neurofilament protein into embryonic stem cells under conditions whereby said nucleic acid becomes integrated into the genetic material of the embryonic stem cell, transferring said embryonic stem cells into blastocysts, implanting said blastocysts into the reproductive tract of a pseudopregnant female recipient, and maintaining said female recipient under conditions whereby genetically engineered offspring are produced.

17. A method of making a genetically engineered non-human animal containing one or more human neurofilament genes comprising introducing a nucleic acid that encodes a human neurofilament gene into embryonic stem cells under conditions whereby said nucleic acid becomes integrated into the genetic material of the embryonic stem cell, transferring said embryonic stem cells into blastocysts, implanting said blastocysts into the reproductive tract of a pseudopregnant female recipient, and maintaining said female recipient under conditions whereby genetically engineered offspring expressing one or more human neurofilament genes is produced.

18. The method of claim 17 wherein the human neurofilament gene is a wildtype gene.

19. The method of claim 17 wherein the human neurofilament gene is a gene encoding a mutant human neurofilament gene.

20. A method for identifying agents useful for treatment or prevention of neurodegenerative disorders comprising administering a test agent to the genetically engineered animal of claim 1 or 7 and assessing the symptoms and progression of the neurodegenerative disorder, wherein an amelioration in symptoms or progression of the neurodegenerative disorder is indicative of an agent useful in the treatment or prevention of a neurodegenerative disorder.
